(19) United States
(12) Patent Application Publication
(10) Pub. No.: US 2024/0294992 A1
(43) Pub. Date: Sep. 5, 2024

(54) ENHANCED DETECTION OF BIOFILM-EMBEDDED AND ADHERED PATHOGENS ON CONTAMINATED FOODS OR SURFACES USING ENZYMES

(71) Applicants: The United States of America, as represented by the Secretary of Agriculture, Washington, DC (US); UNIVERSITY OF VIRGINIA PATENT FOUNDATION, CHARLOTTESVILLE, VA (US)

(72) Inventors: JOSEPH A CAPOBIANCO, JR., MARKETING, NY (US); JOSEPH LEE, FLEMINGTON, NJ (US); CHIN YI CHEN, HUNTINGDON VALLEY, PA (US); YIPING HE, AMBLER, PA (US); CHERYL M ARMSTRONG, ABINGTON, PA (US); SUE A REED, MORGANTOWN, PA (US); BRYAN BERGER, CHARLOTTESVILLE, VA (US)

(21) Appl. No.: 18/358,437

(22) Filed: Jul. 25, 2023

Related U.S. Application Data
(60) Provisional application No. 63/393,071, filed on Jul. 28, 2022.

(51) Int. Cl. C12Q 1/689; C12Q 1/18; C12Q 1/28; C12Q 1/6851

(52) U.S. Cl. CPC C12Q 1/689; C12Q 1/18; C12Q 1/28; C12Q 1/6851

(57) ABSTRACT

Provided herein are compositions and methodologies for identifying microbes in biofilms and adhered to biotic and abiotic surfaces, utilizing enzymes that degrade biofilms and disperse aggregated clusters of microorganisms. Utilizing enzymes such as CAase to degrade biofilms, organisms released from biofilms are identified more readily than from untreated biofilms. Biofilms from a variety of sources, both biotic and abiotic, can be analyzed utilizing the present disclosure.

### CROSS-REFERENCE

The present application claims priority to U.S. Provisional Patent Application Ser. No. 63/393,071 filed Jul. 28, 2022, the contents of which are expressly incorporated herein by reference.

### BACKGROUND OF THE INVENTION

### Field of Invention

Provided herein are compositions and methodologies for identifying microbes in biofilms and microbes adhered to surfaces utilizing enzymes that degrade biofilms. Utilizing enzymes such as CAase to degrade biofilms, organisms released from biofilms are identified more readily than from untreated biofilms. Biofilms and other adherent microbes from a variety of sources, both biotic and abiotic, can be analyzed utilizing the present disclosure.

### Background

Microbes exist largely in a sessile state in biofilms, which encapsulate cells in a matrix of DNA, protein, and polysaccharides (Limoli et al, Microbiol. Spectr., (2015), 3(3): doi:10.1128/microbiolspec.MB-0011-2014). Biofilm-embedded cells are able to adhere to a wide range of surfaces, proliferate and shield themselves from external stresses such as chemical sanitizers, mechanical disruption, heat and ultraviolet (UV) treatment (Bridier et al, Food Microbiol., (2015), 45:167-78). Thus, in a processing environment, biofilm is a major challenge to remove and acts as a primary source of persistent contamination. Biofilm-embedded cells can present several problems, including contamination with foodborne pathogens such as Escherichia coli, Listeria monocytogenes and Salmonella enterica (Zhao et al, RSC Advances, (2017), 7:36670-36683). They can also lead to corrosion of metal surfaces and degradation of processing equipment, in addition to causing spoilage due to the release of proteases, lipases and other enzymes that degrade food products (Dula et al, Folia Microbiol., (2021), 66:293-302; Galié et al, Front. Microbiol., (2018), 9: doi: 10.3389/fmicb.2018.00898). The inability to separate bacterial cells from food and environmental samples hinders the detection of pathogens as well (Rohde et al, Biomed. Res. Int'l., (2015), 145437: doi: 10.1155/2015/145437; Stevens & Jaykus, Crit. Rev. Microbiol., (2004), 30:7-24). Historically, the adsorption of bacteria to food products such as meat and produce has been associated with a decrease in microbial recovery during sample processing for subsequent pathogen detection. Therefore, removal of biofilm is critical to ensuring food safety, with implications for both human health and product quality.

For biofilm removal, some enzymes including proteases, DNases, lipases and polysaccharide-degrading enzymes have all been explored as possible tools for biofilm removal (Gutiérrez, T. J. (2019). “Antibiofilm Enzymes as an Emerging Technology for Food Quality and Safety”, in M. Kuddus (Ed.), Enzymes in Food Biotechnology, (2019):321-342) Academic Press). In the food industry, prior studies have shown that mixtures of these enzymes can be effective at removing adherent biofilm-embedded colonies, and in combination with chelating agents and detergents, can be effective in dislodging biofilms as well as potentially breaking down various components within them such as DNA, protein and polysaccharides (Lequette et al, Biofouling, (2010), 26:421-31). However, many studies have suggested that some enzymes such as amylases may exacerbate spread of biofilm-embedded cells rather than preventing biofilm formation, and application of a specific mixture of enzymes tailored to the biofilm composition and processing conditions are required to fully degrade a specific biofilm (Meireles, Borges, Giaouris, & Simões, 2016).

To meet these needs, we herein provide a novel approach using biofilm-degrading enzymes to enhance identification of biofilm-embedded pathogens.

### SUMMARY OF THE INVENTION

The present disclosure provides a method for identifying microbial cells embedded in a biofilm, by: a) contacting the biofilm with an enzyme for a sufficient time to release microbial cells from the biofilm; b) separating the released microbial cells from the biofilm material; c) collecting the released microbial cells; and d) determining the genus or species of at least one released microbial cell, thereby identifying a microbial cell previously embedded in the biofilm. In some embodiments, the microbe is a bacterium and in specific examples, the bacterium is Listeria monocytogenes or Salmonella Enteritidis. In some embodiments, the enzyme utilized is CAase. In some embodiments, released microbial cells are not contacted with a growth medium after release from the biofilm. In some embodiments, all steps of this method are completed within 6 hours. In some embodiments, the sufficient time to release microbial cells with an enzyme is less than 2 hours.

Also provided herein is a method for identifying microbial cells adhered to a surface, by: a) contacting the surface with an enzyme for a sufficient time to release microbial cells from the surface; b) collecting the released microbial cells; and c) determining the genus or species of at least one released microbial cell, thereby identifying a microbial cell previously adhered to the surface. In some embodiments, the microbe is a foodborne pathogen. In some embodiments, the enzyme utilized is CAase. In some embodiments, the released microbial cells are not contacted with a growth medium. In some embodiments, the surface is a biotic surface, such as meat, a ready-to-eat meat product, or fresh produce. In some embodiments, the surface is an abiotic surface, such as a food preparation surface. In some embodiments, all steps of this method are completed within 6 hours. In some embodiments, the sufficient time to release microbial cells with an enzyme is less than 2 hours.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1A and FIG. 1B depict analysis of the minimum inhibitory concentration of CAase on L. monocytogenes. FIG. 1A: A representative 96-well plate used to assess the MIC of L. monocytogenes. Columns 1-10 contain CAase and 10⁴ CFU/mL of live L. monocytogenes in BHI. CAase concentrations were diluted 2-fold from column 1 to 10 as indicated. CAase was not added to columns 11-12, which served as controls containing either heat-killed L. monocytogenes (column 11) or live L. monocytogenes (column 12). FIG. 1B: Measurement of cell growth (OD₆₀₀) is plotted on the y-axis while CAase concentration (mg/mL) is depicted on the x-axis. The average of 4 trials is shown with error bars representing the standard deviation and groups not connected by the same letter determined to be significantly different (p<0.05).

FIG. 2A, FIG. 2B, FIG. 2C, FIG. 2D and FIG. 2E provide biofilms of L. monocytogenes and crystal violet assay results. FIG. 2A: Glass slides with L. monocytogenes biofilms in a 6-well plate after rinsing and prior to CAase treatment. FIG. 2B: Tissue culture tubes with L. monocytogenes biofilms after rinsing and prior to CAase treatment. FIG. 2C: Glass slides with L. monocytogenes biofilms after treatment and crystal violet staining. FIG. 2D: Tissue culture tubes with L. monocytogenes biofilms after treatment and crystal violet staining. Visual inspection of the samples after staining with crystal violet clearly demonstrate a substantial reduction in biofilm in the CAase-treated samples compared to PBS samples. FIG. 2E: The graph depicts the results of the crystal violet assay. Adsorption at 590 nm is plotted on the y-axis and the treatments, grouped by sample, are presented on the x-axis. Error bars were constructed using 1 standard deviation from the mean. The picture inserts display representative crystal violet solutions from these experiments. The absorbance measurements for CAase and PBS treatments were observed to be different for both culture tubes (p<0.001) and glass cover slips (p=0.0118).

FIG. 3A, FIG. 3B, FIG. 3C, and FIG. 3D provide scanning electron microscope images of L. monocytogenes biofilms. FIG. 3A: PBS treated biofilms at 2500×. FIG. 3B: CAase treated biofilms at 2500×; FIG. 3C: PBS treated biofilms at 10,000×. FIG. 3D: CAase treated biofilms at 10,000×.

FIG. 4A and FIG. 4B provide graphical representation of CAase treatment affecting the detection of Listeria biofilms adhered to glass surfaces. FIG. 4A: Quantitation of cells released into the solution as determined by the 6×6 culture plating method. Calculated CFU/mL from the 6×6 plating method is plotted as a function of time with error bars representing one standard deviation from the mean. FIG. 4B: An assessment of the release of cells into solution via qPCR was plotted with the y-axis depicting Ct and the x-axis depicting time. Error bars represent one standard deviation from the mean.

FIG. 5A, FIG. 5B, and FIG. 5C provide pictorial and graphical representation of detection of CAase-treated Listeria biofilms grown on RTE Meat. FIG. 5A: Image of PBS-treated RTE meat. FIG. 5B: Image of CAase-treated RTE meat; FIG. 5C: A qPCR assessment of the cells released into solution from RTE samples 6 hours post CAase treatment with the Ct values for various simulated levels of L. monocytogenes contamination (CFU/mL of inoculum) being plotted. The error bars represent one standard deviation from the mean. The corresponding results from the 3M MDS analysis are presented as an inset; a plus sign (+) indicates detection of L. monocytogenes contamination in 2 independent samples, a plus and minus sign (+,−) indicates positive detection in 1 of the samples and negative detection in another, and a minus sign (−) indicates L. monocytogenes was not detected in either independent replicate.

FIG. 6A and FIG. 6B depict analysis of the minimum inhibitory concentration of CAase on Salmonella enterica serovar Enteritidis (Salmonella Enteritidis). FIG. 6A: A representative 96-well plate used to assess the MIC. FIG. 6B provides graphical representation of the minimum inhibitory concentration of CAase on Salmonella enterica serovar Enteritidis (Salmonella Enteritidis).

FIG. 7 provides graphical representation of CAase treatment affecting the detection of Salmonella Enteritidis biofilms adhered to glass surfaces.

FIG. 8A and FIG. 8B provide scanning electron microscope images of Salmonella Enteritidis biofilms. FIG. 8A: CAase-treated biofilms at 2500×. FIG. 8B: PBS-treated biofilms at 2500×.

### DETAILED DESCRIPTION OF THE INVENTION

The present disclosure provides compositions and methods for increasing efficiency of identifying biofilm-embedded organisms. Such methods generally include the steps of exposing a biofilm to a biofilm-degrading enzyme, collecting the cells released from the biofilm, and identifying the cells.

As reported herein, we tested the ability of enzymes, including an engineered polysaccharide degrading enzyme (“CAase”; see, e.g., U.S. patent application Ser. No. 17/306,623) to facilitate identification of biofilm-embedded organisms and organisms adhered to surfaces. CAase has been reported as having broad-spectrum ability to degrade biofilms associated with a wide range of gram-negative (E. coli O157:H7, Salmonella) and gram-positive (L. monocytogenes) pathogens (Mayton et al, Appl. Environ. Microbiol., (2021), 87:e0026521). The ability of a single enzyme to degrade a wide range of biofilms to release biofilm-embedded cells and prevent adhesion is distinct from many other enzyme-based approaches, in which enzyme mixtures, often with other chelating agents and detergents added, are required. We further investigated the impact of multiple enzymes, including CAase, on biofilm formation and cell growth and determined conditions under which inhibition of biofilm formation and cell growth occur.

We have identified novel functions and methodologies using minimal enzyme concentrations for biofilm removal from abiotic (glass) and biotic (ready-to-eat meat) samples and demonstrate that enzyme treatment enhances detection of bacteria, such as the exemplary organisms L. monocytogenes, E. coli and S. enterica. Such a novel approach can be used, for example, to reduce the number of cycles required for qPCR-based detection and can be used to identify pathogens at lower contamination levels using other standardized tests, compared to untreated samples. Collectively, these results indicate the effectiveness of biofilm-degrading enzyme treatment in lowering the threshold of detection of biofilm-forming pathogens in ready-to-eat (RTE) products and abiotic surfaces.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms “isolated”, “purified”, or “biologically pure” as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

The term “consisting essentially of” excludes additional method (or process) steps or composition components that substantially interfere with the intended activity of the method (or process) or composition and can be readily determined by those skilled in the art (for example, from a consideration of this specification or practice of the invention disclosed herein).

The term “biofilm” as used herein refers to a composition or matrix of microorganism strains/species formed on either a biotic or abiotic surface. Biofilms also typically include microorganisms encapsulated in the matrix which generally containing proteins, nucleic acids and exopolysaccharides as the main components.

“Biofilm degrading enzyme”, and grammatical variants thereof, means enzymes capable of depolymerizing or degrading the components of a microbial biofilm. Such enzymes include, but are not limited to, DNases, restriction endonucleases, dispersin B, glycoside hydrolases, proteases, and CAase.

The term “degrade”, with reference to biofilms, means to reduce, liberate, or disperse biomass and matrix components thereof that are part of or associated with the biofilm when compared to another condition or control. In the context of the instant application, “degrading” a biofilm is achieved via the use of enzymes, although other active and inactive components can also be utilized.

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The term “plant pathogen” as used herein refers to any disease-causing microorganism carried by an insect pest, transmitted to, or otherwise acquired by, a tree or plant or its harvest products (e.g., fruit, citrus, nuts) that causes harm to the tree or plant or its harvest products resulting in economic loss to the agricultural industry. Routes of transmission and acquisition of a plant pathogen can include, but are not limited to, insect pest carriers, open wounds (cuts, punctures, abrasions, foraging damage), agricultural waters, biosolids, and storage surfaces.

As used herein, “foodborne pathogen” refers to a microorganism such as a bacterium, a protist, or a fungus capable of contaminating food, food contact surfaces, food processing surfaces, food processing equipment, or process water and causing disease to humans or animals ingesting or contacting the food.

The term “fresh produce” as used herein refers generally to farm-produced fruits and vegetable crops including, but not limited to fruit and vegetable crops such as e.g., corn, bean, and cilantro, cabbage, almonds, cucumbers, cantaloupes, etc.

### Adhesion and biofilms

Adsorption, or the assimilation of dissolved matter by a surface, is mediated by nonspecific and reversible physicochemical interactions including Van derWaal's forces, electrostatic interactions, hydrophobic interactions, and hydrogen bonding (Zárate et al, J. Food Prot., (2002), 65:534-9; Lukasik et al, J. Food Prot., (2001), 64:292-7; Glantz et al, Acta Otontol. Scand., (1999) 57:5). Bacterial adsorption to surfaces is mediated by various constituents including teichoic acids, proteins, nucleic acids and carbohydrate moieties, and can include assembly of above components into subcellular structures such as fimbriae or flagella to facilitate attachment and adhesion (Meylheuc et al, J. Appl. Microbiol., (2001), 91:822-32). Biofilms are a special case of bacterial adsorption which are composite structures consisting of biologically active microorganisms and associated extracellular polymeric materials which encapsulate cells to adhere to surfaces. (Kumar & Anand, Int'l. J. Food Microbiol., (1998), 42:9-27. Bacterial adhesion in biofilms is affected by numerous variables including nutrient availability, type and growth stage of the microorganism(s), pH, temperature, and the nature of the surface itself. The forces involved in biofilm formation are like those involved in bacterial adsorption described earlier, and include hydrophobic interactions, dipole-dipole interactions, and ionic and covalent bonding (Stevens & Jaykus, Crit. Rev. Microbiol., (2004), 30:7-24).

Biofilm is a naturally occurring state of microorganisms, in which cells come in contact with and adhere to a surface. This sessile state of microorganisms is common and distinct from a planktonic state in which individual cells migrate. In a biofilm, microorganisms synthesize DNA, proteins, lipids and polysaccharides, which encapsulate cells and facilitate attachment of a colony of individual cells, often referred to as an ‘immature’ biofilm. The composition of biofilm often changes as this colony grows to become a ‘mature’ biofilm. A biofilm community can contain a single or multiple distinct microorganisms, each of which contributes to generation of the biofilm. Secreted exopolysaccharides (EPS) are the major components found in the majority of microbial biofilms.

### Biofilm-Targeting Enzymes

Biofilms are generally composed of an extracellular polymeric matrix. Biofilm matrices typically contain proteins, polysaccharides, nucleic acids, and lipids. Enzymes that degrade biofilm matrix components can be utilized for the methodologies and compositions disclosed herein, as long as the enzyme effects a release of microorganisms from the biofilm. This includes proteases to target proteins present in biofilm, exo- and endonucleases to degrade nucleic acids present in biofilm, lipases to degrade lipids present in biofilm, and enzymes targeting expolysaccharides present in biofilm. Dispersin B is one such example, which degrades the polysaccharide poly-N-acetyl-glucosamine. Another example is CAase, which degrades polysaccharides present in mature microbial biofilms. Polysaccharide lyases represent another category of enzymes that degrade biofilms containing uronic acid components such as mannuronic acid found in alginate and related biofilms; examples include AlgL from P. aeruginosa and other gram-positive/gram-negative bacteria and fungi with differing specificities for particular uronic acids present in the biofilm polymers. Glycosyl hydrolases are a large category of enzymes that degrade a diverse range of polysaccharides found in biofilms; examples include amylases, which decomposes starches to sugars such as maltose, glucose and dextrins, cellulases, which depolymerize cellulose polymers to produce glucose. Related to glycosyl hydrolases are enzymes that act on peptidoglycan and other cell wall polysaccharides, which include muramidases such as lysozyme that act on major cell wall peptidoglycan polysaccharides and lysins, which degrade murein present in bacterial cell wall peptidoglycan. Lipases act to degrade lipids and lipopolysaccharides present in biofilms and cell walls, thereby disrupting biofilm structure. Proteases are also used to degrade secreted proteins present in the biofilm matrix, and include trypsin, chymotrypsin, and proteinase K. Nucleic acid degrading enzymes are also used to degrade extracellular DNA included in biofilm; one example is Pulmozyme, which is used therapeutically to reduce solution viscosity due to biofilm formation in cystic fibrosis patients.

### Biofilm-Forming Organisms

The methodologies provided herein can be used with any biofilm-forming organisms, including bacteria, archaea, fungi and microalgae. Additionally, biofilms containing and/or formed by multiple organisms are subject to the methodologies herein. Exemplary biofilm-forming organisms include, but are not limited to, Listeria monocytogenes, Lactobacillus spp. (e.g., Lactobacillus plantarum), Campylobacter spp. (e.g., C. jejuni), Lactococcus spp. (e.g., Lactococcus lactis), Escherichia coli, Pseudomonas spp. (e.g., P. aeruginosa, P. putida), Salmonella spp. (e.g., S. enterica), Agrobacterium spp. (e.g., A. tumefaciens), Aeromonas spp., Erwinia spp. (e.g., E. amylovora), Stenotrophomonas spp. (e.g., S. maltophilia), Acinetobacter spp. (e.g., A. baumannii), Achromobacter spp. (e.g., A. xylosoxidans), Pandoraea spp. (e.g., P. apista), Burkholderia spp. (e.g., B. cepacia), cyanobacteria, Clavibacter michiganensis, Aspergillus spp. (e.g., A. nidulans), Cryptococcus spp. (e.g., C. laurentii, C. neoformans), Candida spp. (e.g., C. albicans, C. parapsilosis, C. glabrata), Botyrtis spp. (e.g., B. cinerea), Fusarium spp. (e.g., F. oxysporum), microalgae, diatoms, Pantoea spp. (e.g., P. stewartii, P. agglomerans), Ralstonia solanacearum, Staphylococcus spp. (e.g., S. aureus), Streptococcus spp. (e.g., S. mutans), and Xanthomonas spp. (e.g., X. axonopodis, X. campestris, X. oryzae).

The embodiments illustratively disclosed herein may be suitably practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

Having generally described this invention, the same will be better understood by reference to certain specific examples, which are included herein to further illustrate the invention and are not intended to limit the scope of the invention as defined by the claims.

### EXAMPLES

### Example 1

### Analysis of CAase for Identifying Biofilm-Embedded L. monocytogenes

### General Methods and Conditions

Nanopure water was obtained from a Barnstead ultrapure water purification system (Thermo Fisher Scientific, Inc., Canoga Park, CA). Piranha solution was prepared by mixing a 3:1 volume ratio of sulfuric acid (Sigma Aldrich) and 30% hydrogen peroxide (Sigma Aldrich). Oxoid Brain Heart Infusion (BHI) Broth (Thermo Fisher Scientific) was prepared by dissolving 37 g in 1 L of nanopure water. 1.5% agar (Becton Dickinson) was added to the broth solutions to prepare plates. Media were prepared according to the manufacturer's recommendations. Crystal violet solution, 1%, was purchased from Sigma Aldrich and diluted with nanopure water. CAase was expressed and purified using previously published methodology (Mayton et al, supra).

Isolated colonies of Listeria monocytogenes ATCC 19111 were used to inoculate 25 mL of BHI Broth. This L. monocytogenes strain was chosen because of its ability to produce a robust biofilm, known to remain intact even after repeated wash/rinse cycles. Cultures were aerobically grown overnight at 30° C. with shaking at 200 rpm (Innova 42, New Brunswick, Enfield, CT). The overnight culture was adjusted to an OD₆₀₀ of 1.0 (˜10⁹ CFU/mL) and serial dilutions were subsequently prepared using BHI. Suspensions of heat-killed cells were prepared using a thermomixer at 99° C. for 10 minutes and then cooled to room temperature.

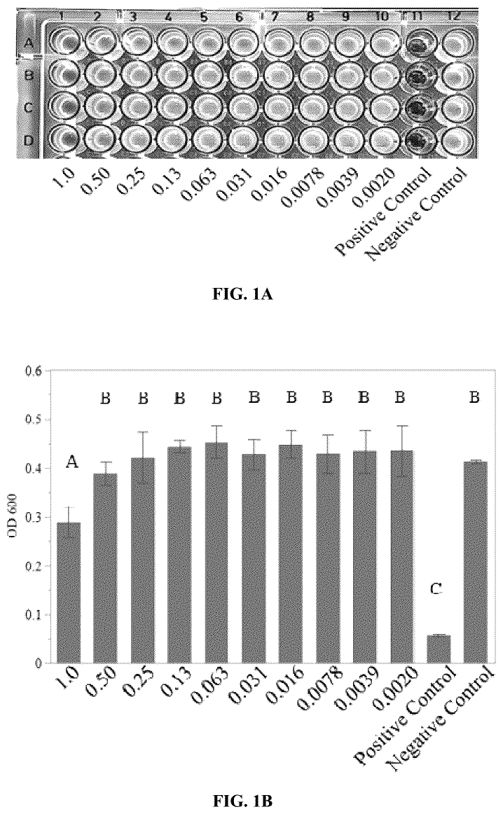

The minimum inhibitory concentration (MIC) of CAase was determined using a 96-well plate format with a total of 200 μL of solution being placed into the individual wells (FIG. 1A). Experimental solutions (columns 1-10) were composed of BHI, purified CAase (with concentrations ranging from 0.00195-1.0 mg/mL), and L. monocytogenes (10⁴ cells/well). Control solutions were composed of BHI with either heat-killed L. monocytogenes (column 11) or live L. monocytogenes (column 12) but did not contain CAase. Plates were incubated overnight (˜20 hours) at 30° C. and the absorbance was measured at 600 nm using a TECAN SAFIRE² microplate reader (Männedorf, Switzerland).

Circular glass coverslips with diameter of 12 mm (Electron Microscopy Sciences, Hatfield, PA) or 22 mm square glass coverslips (Corning) were cleaned with a 3:1 piranha solution for 30 minutes. The slides were rinsed twice with 50 mL of sterile nanopure water, dried under aseptic conditions, and stored under nitrogen prior to use.

Biofilms were grown on glass slides as previously described (Reis-Teixeira, Alves, & de Martinis, 2017). Briefly, slides were seeded under static conditions at 25° C. in 5 mL of an overnight culture of L. monocytogenes. After 3 hours, the slides were gently rinsed with BHI broth and then transferred to tissue culture tubes (TPP Techno Plastic Products AG, Switzerland) containing 5 mL of BHI broth. The samples were subsequently incubated at 25° C. at 120 RPM for 8 days to allow for the growth of a mature biofilm. Slides were then washed three times in 50 mL of PBS, subjected to CAase treatment, and assayed using crystal violet staining, and/or microbial plating and real-time PCR.

A 5 oz ready-to-eat smoked summer sausage was purchased from a local supermarket and aseptically cut into 0.55 g cylindrical samples. The sample was sliced to a thickness ranging from 4 to 6 mm and then cut with a #8 corkborer to ensure the diameter was similar to that of the glass slides, 12 mm. The cylinders were weighed to select samples that would utilize the ratio of sample to media (25 g/225 mL) used in the Microbiology Laboratory Guide method number 8 (United States Department of Agriculture Food Safety and Inspection Service & Science, 2021).

Biofilms were grown on the sausage samples using the protocol described for glass with one significant difference. All glass surfaces were seeded with overnight cultures, whereas a serial dilution of an overnight culture was used to prepare biofilms for the sausage experiments to simulate varying degrees of contamination on the meat. Using 5 mL of the serial dilution 0.55 g samples were seeded with a concentration of L. monocytogenes ranging from 10⁷ CFU/mL to 10² CFU/mL. Samples that were not inoculated served as negative controls.

Biofilm samples were then transferred to individual wells of 12-well plates or 15 mL conical tubes. Surfaces with L. monocytogenes biofilms were treated with 5 mL PBS, or 5 mL of a 0.1 mg/mL CAase solution in PBS. The samples were incubated at 25° C. with shaking at 30 RPM. To evaluate the removal of cells from the biofilm, the liquid above the surface onto which the biofilm was attached (glass or RTE meat) was collected and analyzed via bacteria plating and/or molecular characterization. During the time course trials, equivalent volumes of PBS or 0.1 mg/mL CAase were added after aliquots were collected to ensure a consistent volume throughout the duration of the experiment.

The glass slides were transferred to a 6-well plate where they were submerged in 5 mL of PBS or 0.1 mg/mL CAase as previously described. The volume of PBS and 0.1 mg/mL CAase was increased to 7 mL to treat culture tubes to ensure the volume would cover the biofilm ring. The samples were incubated at 25° C. with shaking at 30 RPM for 6 hours. After 6 hours, the tubes and glass slides were rinsed 3 times with PBS.

The culture tubes were treated with 7 mL of a 0.1%(w/v) solution of crystal violet and the glass slides were treated with 5 mL of the same solution. After 30 minutes, the crystal violet solution was removed, and the samples were rinsed 3 times with PBS. The culture tubes were then treated with 7 mL of 95% ethanol while the glass slides were treated with 5 mL of the same solution. The culture tubes and glass slides were scraped, and the dispersed suspensions were permitted to incubate at room temperature for 30 minutes in individual wells of a 6-well plate. The glass slides were then removed from each well of the 6-well plate, and the absorbance value was measured at 590 nm for each well in a TECAN SAFIRE² plate reader. The solution from each culture tube was dispensed into 16 wells in a transparent 96 well plate; each well contained 200 μL of solution. The absorbance was measured using a wavelength of 590 nm in as TECAN SAFIRE² plate reader.

Aliquots of 200 μL samples collected at 0, 0.5, 1, 2, 3, 4, 5 and 6 hours after the introduction of CAase or PBS were enumerated according to a 6×6 drop method previously described, except 7 μL per drop was used/12 drops per dilution (Chen et al, J. Microbiol. Meth., (2003), 55:475-79). Colonies were counted after overnight incubation at 30° C. using a dissecting microscope and the concentration of bacteria (CFU/mL) was calculated. All microbiological plating assays were conducted in quadruplicate.

Samples collected at 0, 0.5, 1, 2, 3, 4, 5 and 6 hours after the introduction of CAase or PBS were subjected to qPCR as previously described (Suo et al, Foodborne Path. Dis., (2010), 7:619-28). Briefly, 200 μL samples were centrifuged at 6,600×g for 10 minutes. A pellet was not visible in all the samples so 180 μL of supernatant was removed and 20 μL of PrepMan Ultra Reagent (Applied Biosystems, Rotkreuz, Switzerland) was subsequently added before statically incubating samples at 100° C. for 10 minutes. Samples were centrifuged again for 10 minutes at 6600×g and 15 μL of supernatant containing DNA was collected. The qPCR assay contained a previously reported internal amplification control (IAC) and was performed using a 7500 Real-Time PCR System (Applied Biosystems). 2 μL of each sample was added to a well in a 96-well plate containing an 18 μL mixture of 1×TaqMan Gene Expression Master Mix (Applied Biosystems), two sets of primers (hlyA and IAC) and probes (FAM and TAMRA) with a concentration of 200 nM each, 1.2×10⁴ copies of IAC and ddH₂O. The qPCR assays were performed in triplicate using the published thermocycling protocol. The measured responses were analyzed using automatic Ct and automatic baseline settings in Applied Biosystems 7500 System software v1.4 and exported to JMP for analysis.

Samples of 200 μL each collected 6 hours post treatment with PBS or CAase were analyzed via the Molecular Detection Assay—Listeria monocytogenes (3M, St. Paul, MN) using the manufacturer's instructions without enrichment. The data was analyzed using 3M MDS software (Version 2.3.0.1).

Biofilms grown on glass coverslips were fixed in 2.5% glutaraldehyde and prepared for SEM imaging using previously described methods (Xie et al, Appl. Environ. Microbiol., (2011), 77:2325-31). All SEM images were collected using a Quanta 200 FEG scanning electron microscope (FEI, Hillsboro, OR). All the quantitative data was analyzed and graphically presented using JMP software version 14.3.0.

### CAase Minimum Inhibitory Concentration (MIC)

CAase has demonstrated an ability to remove biofilms produced by various gram-negative and gram-positive pathogens, but also appeared to affect the hydrophobicity of the cells (Mayton et al, supra). To determine if the reduction in biofilm was related to cell death, we examined the MIC of the enzyme for L. monocytogenes. Here, the MIC was defined as the lowest concentration of additive that completely inhibited visible growth after an overnight incubation. Visual inspection of the plate (FIG. 1A) determined the MIC for CAase to be >1.0 mg/mL, which was beyond the range tested in this report. Student's t-tests comparing the optical density measurements at 600 nm (OD₆₀₀) of live cells treated with CAase and heat-killed cells substantiated this conclusion (p<0.0001 for comparing all levels) (FIG. 1B). Furthermore, OD₆₀₀ values obtained from samples with CAase concentrations ranging from 0.00195 to 0.5 mg/mL did not differ significantly from those obtained in the absence of CAase (p>0.1070). However, OD₆₀₀ values for CAase treatments greater than 0.5 mg/mL are significantly different from those obtained with lower concentrations of CAase (p<0.008), suggesting that this concentration may be approaching the MIC. As a result of these findings, an enzyme concentration of 0.1 mg/mL was utilized throughout the remainder of this study since it was determined that the use of CAase at this concentration did not inhibit the growth of L. monocytogenes.

The results demonstrate that enzyme (CAase) treatment can remove biofilm at enzyme concentrations that do not appear to inhibit cell growth since significant reductions in biofilm formation were seen using 0.1 mg/mL CAase whereas inhibition of L. monocytogenes growth was observed at concentrations above 0.5 mg/mL CAase (FIG. 1A). These results are consistent with a mechanism by which CAase targets specific exopolysaccharide (EPS) components present in L. monocytogenes biofilm rather than polysaccharides important for cell-wall integrity and growth.

### CAase Treatment Effects on Biofilm Structure and Morphology

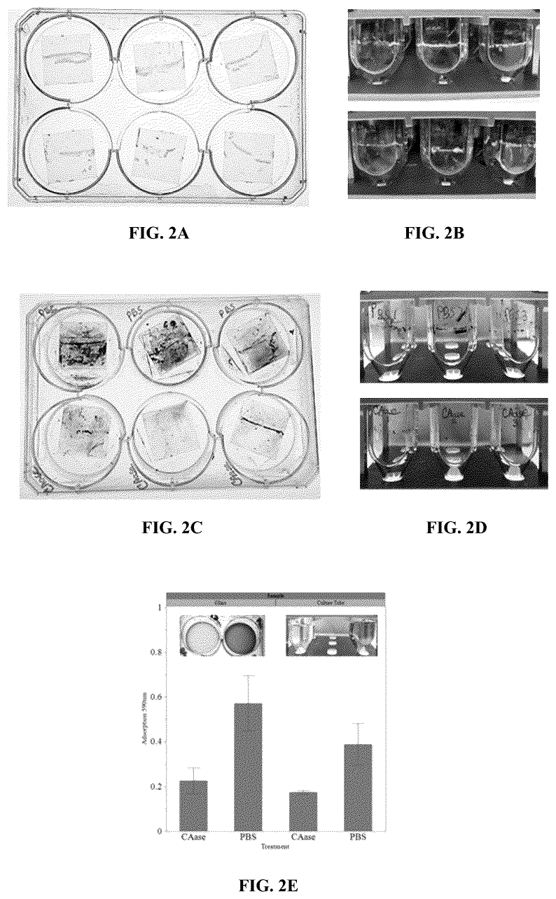

L. monocytogenes ATCC 19111 was grown on glass surfaces and in plastic tissue culture tubes. Prior to treatment, deposition of adherent biofilm was visible to the naked eye on both glass slides incubated in cell suspensions (FIG. 2A) and at the air-liquid interface of liquid cultures grown in plastic culture tubes (FIG. 2B). Next, the ability of the CAase enzyme to reduce L. monocytogenes biofilm formation on both the glass slides and the plastic culture tubes was determined using a 6-hour treatment period with 0.1 mg/mL CAase or a PBS control using gentle agitation. Biofilm formation following PBS or CAase treatment was both visually detected (FIG. 2C and FIG. 2D) and quantified using a crystal violet staining assay FIG. 2E. A 50-70% reduction in biofilm was seen for both culture tubes and glass slides treated with CAase compared to the PBS control, which was statistically significant for both culture tubes (p<0.001) and glass cover slips (p=0.0118).

Prior studies have determined the chemical structure of EPS produced by L. monocytogenes is comprised of a repeating b-1,4-linked N-acetylmannosamine chain with terminal a-1,6-galatose linkages and have also identified a recombinant form of a catalytic domain within a transmembrane glycosylhydrolase (PssZ) that was capable of dispersing biofilm-embedded cell aggregates in solution at approximately 1-30 mg/mL, but was unable to disperse ethanol-purified EPS in vitro (Köseoğlu et al, supra). The results presented herein are consistent with these observations, suggesting CAase degrades EPS by the observed reduction in crystal violet staining at similar concentrations (100 mg/mL) to that observed using PssZ.

### Scanning EM Analysis of Treated Biofilms

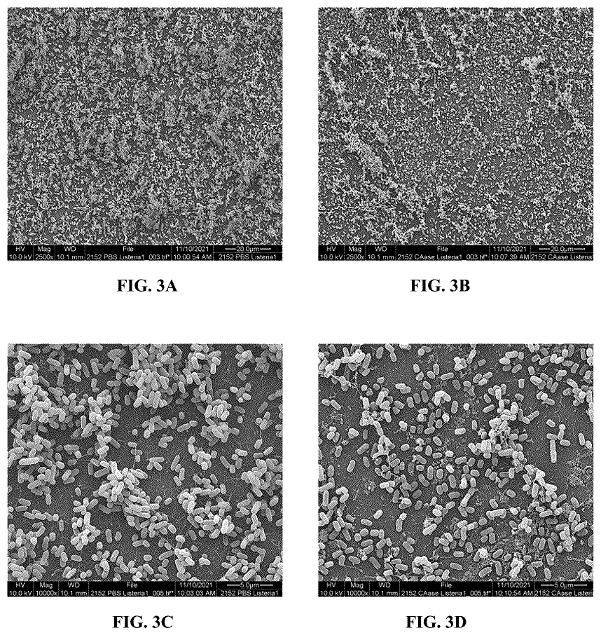

To understand how CAase may be affecting biofilm structure and morphology, biofilms were grown on glass coverslips followed by CAase or PBS treatment for 6 hours, then glutaraldehyde fixed and imaged using scanning electron microscopy (SEM). At 2,500× magnification, the buffer-only control showed distinct 3-D clusters of cells present on the glass surface throughout each image (FIG. 3A), whereas samples treated with CAase had overall fewer cells present and fewer clusters of cells (FIG. 3B). At 10,000× magnification, the disruption of individual cell clusters was more apparent; the CAase treated samples showed overall fewer cells and diminished 3-D cell clusters (FIG. 3D) compared to PBS control (FIG. 3C). Consistent with our MIC and biofilm measurements, no significant changes in cell wall integrity were observed. Thus, the SEM results provide further evidence to support a primary role for CAase in removing adherent biofilm of L. monocytogenes.

Further evidence to support degradation of L. monocytogenes EPS comes from direct observation of cultures treated with CAase at sub-MIC concentrations (FIG. 3B), in which CAase treatment causes disaggregation of cell clusters and fewer interconnected chains of bacterial cells. Prior studies on L. monocytogenes biofilm formation indicated that under biofilm conditions, organized clusters of cells were connected by chains of individual cells, and the agr operon was upregulated in cells present in the individual chains outside of cell clusters present in biofilm as well as early stages of biofilm formation (Rieu et al, Appl. Environ. Microbiol., (2008), 74:4491-97). Similar biofilm morphologies were observed for untreated cells (FIG. 3A), whereas these structures are degraded and removed with sub-MIC CAase treatment. Aggregation of L. monocytogenes cells into clusters is attributed to EPS production, and therefore addition of CAase to cultures to remove total biofilm EPS (FIG. 2A) as well as the disaggregation of individual cell clusters (FIG. 3B) provides multiple, independent results indicating L. monocytogenes biofilm removal occurs with CAase treatment and is consistent with EPS degradation. Given that L. monocytogenes biofilm formation is linked to higher levels of resistance to antimicrobials and sanitizers and the observed broad-spectrum activity of CAase against Gram-positive and-negative foodborne pathogens, use of CAase as a treatment in food processing environments may enhance the broad-spectrum removal of multiple foodborne pathogens, particularly when used in combination with other conventional chemical sanitizers and disinfectants.

### Improved Detection of L. monocytogenes from Liquids and Solids

Following the observation of significantly reduced biofilms with CAase-treated L. monocytogenes samples and the dissolution of adherent cell clusters into individual cells, investigations into the ability of the enzyme to facilitate detection were pursued. To assess the potential for the CAase enzyme to improve foodborne pathogen detection, enzyme treatment was applied to L. monocytogenes biofilms grown on an inert surface (glass coverslips), as well as on a RTE meat product. Treatment was followed by a traditional microbiological plating method and qPCR-based detection assay of the cells released into the solution to determine if the removal of the biofilm and dislodging embedded cells could enhance detection of L. monocytogenes. For detection, we followed Microbiology Laboratory Guidebook 8 (MLG8) from the USDA Food Safety and Inspection Service (FSIS), which describes methods for detection of Listeria monocytogenes from meat and other sources (United States Department of Agriculture Food Safety and Inspection Service & Science, 2021).

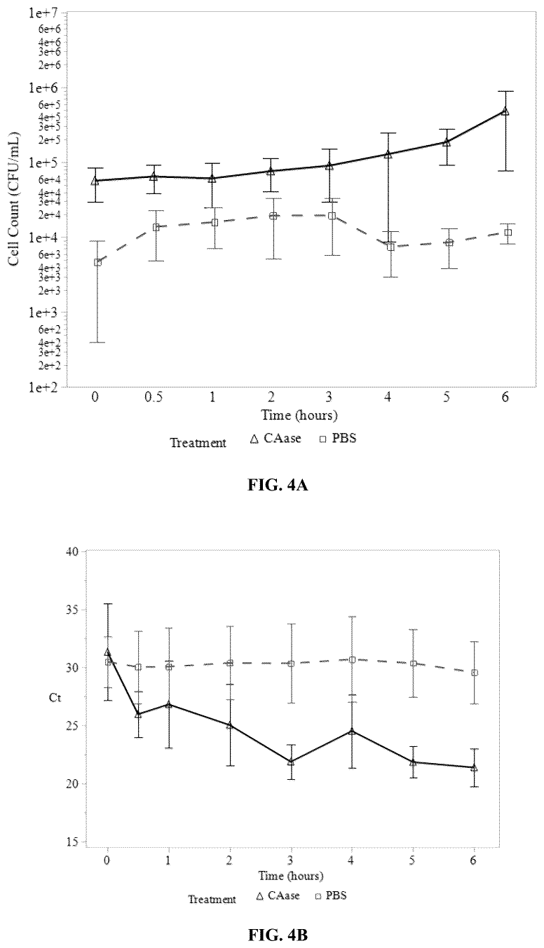

Results from the 6×6 bacterial plating assays conducted are presented in FIG. 4A. For PBS-treated biofilms grown on glass surfaces, the concentration of cells released into the solution slightly oscillates over time (FIG. 4A-dashed line, square points). In contrast, the concentration of cells released into the solution from biofilms on glass surfaces appeared to increase by 4 hours post CAase-treatment (FIG. 4A-solid line, triangle points). Although the concentrations of cells within the solution does not appear to differ for the first 3 hours post treatment (p>0.2566), statistical differences were seen in the measured concentrations at t=4, 5 and 6 hr compared to t=0 using a student's t-test (p=0.0001 for t=5 and 6 hours; p=0.0257 for t=4 hours). In addition, results of the student's t-tests revealed that additional treatment time with CAase led to an increased concentration of cells in solution since a comparison with t=6 hours with either t=5 or t=4 generated a p-value of 0.0001. For completeness, it is important to note that the results of a student's t-test do not indicate that t=4 differs from t=5 (p=0.0714).

Results from the qPCR-based assay used to detect L. monocytogenes are presented in FIG. 4B. From a qualitative inspection of the graph, it appears that when samples are grown on glass coverslips, CAase treatment (0.1 mg/mL) led to a substantial change in the number of cycles (cycle threshold, Ct) detected (or determined) by qPCR. Ct values declined from 30 cycles for the PBS control to 22 cycles with the addition of enzyme, indicating more cells were released into the measured solution from biofilms. Additionally, differences between the number of cells released into solution from PBS versus CAase-treated surfaces were significant based upon Student's t-tests at time intervals t=3, 5, and 6 hr (p=0.0170, 0.0102 and 0.0105 respectively). Note, at t=4 hours, the difference in Ct was not significantly different from t=0 (p=0.0914) due to large SD. Conservatively, the data indicates that CAase treatment is effective beyond 5 hours and that longer periods of exposure could potentially further increase the amplitude of the measured responses.

RTE meat samples inoculated with L. monocytogenes at a range of cell counts (10²-10⁷ CFUs/mL) were used to assess the ability of enzyme treatment to liberate cells from surfaces using qPCR. For additional confirmation of the qPCR results, samples were also assessed using the 3M Molecular Detection System (MDS), a method currently used by USDA Food Safety and Inspection Service for testing and detection of L. monocytogenes in commercial facilities. Independently, we measured the number of qPCR cycles (Ct) required for detection at a given cell count. CAase-treated samples at a given cell count were compared to the PBS control to determine the specific effect of added enzyme on detection. A 6-hour treatment time was selected based upon the collective response from our SEM imaging, microbiological (plating), and qPCR assays that indicate disruption of L. monocytogenes biofilms on glass surfaces.

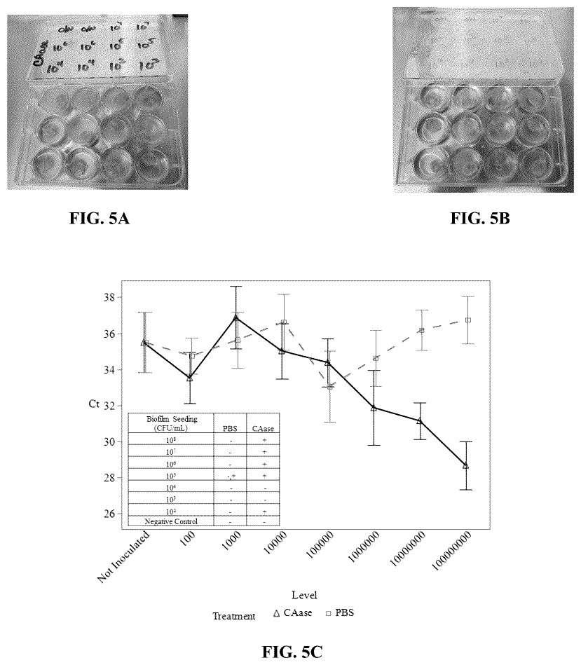

FIG. 5A illustrates RTE meat samples treated with PBS buffer. FIG. 5B illustrates RTE meat samples treated with CAase. FIG. 5C illustrates the results of qPCR assays conducted on cells released into solution from RTE meat samples treated for 6 hours with PBS (FIG. 5A) or CAase (FIG. 5B) that were inoculated with varying concentrations of L. monocytogenes. The non-inoculated Ct value, 35.48, was calculated using the average Ct value for non-inoculated samples determined to be negative by 3M MDS. Like the results seen for glass coverslips, there was a significant reduction in the number of cycles required to detect L. monocytogenes post CAase-treatment (28 cycles) versus the PBS control (36 cycles). Using a Student's t-test, the Ct values for each concentration was compared with that of the non-inoculated samples. For PBS-treated samples, the Ct values at which detection was indicated were not statistically different from the non-inoculated controls (p>0.1404) except for those inoculated with 10⁵ (p=0.0054). This sample was also determined to be positive by the 3M MDS assay. Using a student's t-test the Ct values for samples treated with CAase, inoculant levels of 10⁸, 10⁷ and 10⁶ were determined to be statistically different from non-inoculated samples (p<0.001). These samples were also determined to be positive by the 3M MDS assay. One of the meat samples inoculated with 10² tested positive by the 3M MDS. The Ct value, 33.51, was significantly different from the non-inoculated sample when compared using a Student's t-test (p=0.0248). Thus, it was concluded that the addition of CAase can improve the detection of biofilm-embedded L. monocytogenes on glass coverslips and meat samples, both in terms of increasing the number of cells detected as well as reducing the number of cycle thresholds (and thus time) needed for detection.

These results also point to the potential for enhancing pathogen detection on abiotic and food surfaces using CAase. At a sub-MIC concentration (0.1 mg/mL), the results demonstrated both an effective removal of L. monocytogenes biofilm and a substantial reduction in the number of cycles required for qPCR detection of the culture supernatant (FIG. 4B). For ready-to-eat (RTE) meat samples inoculated with L. monocytogenes (FIG. 5C), buffer-only treated samples were indistinguishable from a non-inoculated control except for a single sample, which yielded a positive result by the 3M MDS assay currently used by USDA FSIS for detection in one of two cases. In contrast, addition of CAase led to consistent detection from 10⁵-10⁸ CFU/mL samples, with one additional positive case out of two for the 10² CFU/mL sample. Further validation of the ability of CAase to improve detection was demonstrated using qPCR with the number of cycles required for detection of samples initially inoculated with 10⁵ CFU/mL and treated with CAase being significantly lower (Ct=28) compared to the buffer-only control (Ct=36). Thus, these results are consistent with the hypothesis that the addition of the biofilm-degrading enzyme CAase releases L. monocytogenes cells, making them available for detection by downstream platforms (such as by a 3M MDS assay) compared to controls where no consistent detection was observed. Processes such as stomaching and blending used in sample pre-processing prior to detection dislodge bacteria from food surfaces; however, dislodged bacteria may not stay in suspension (Hannah et al, Poult. Sci., (2011), 90:491-3). The addition of CAase in sample pre-processing could ultimately increase the number of bacteria present in suspension and relax or minimize the need for culture enrichment prior to detection. Due to the high mortality rate associated with L. monocytogenes infection and the need to detect the presence of even a single bacterial cell, methods that increase detection capabilities are of high importance. Thus, these results provide evidence to support the use of CAase to enhance detection in RTE products, which serve as one of the most significant sources for L. monocytogenes infection (Kurpas et al, J. Vet. Res., (2018), 62:49-55).

Overall, the results of this study demonstrate that use of a polysaccharide-degrading enzyme (CAase) was effective in dislodging adhered bacteria from surfaces removal of the foodborne pathogen L. monocytogenes. Effectiveness of removal was observed for both adherent cultures on abiotic surfaces as well as in RTE products. Interestingly, significant biofilm removal was observed at sub-MIC concentrations, suggesting the primary effect of CAase treatment was the degradation of secreted EPS components found in biofilm. The enzymatic dissociation of cells from biofilm-embedded clusters improved detection via qPCR for both adherent cultures and in RTE foods, reducing the number of cycles and time necessary for detection.

### Example 2

### Analysis of CAase for Identifying Biofilm-Embedded Salmonella Enteritidis

### General Methods and Conditions

Luria Bertani (LB) Broth (Becton Dickinson Co., Sparks, MD) was prepared by dissolving 25 g in 1 L of nanopure H₂O. 1.5% agar (Becton Dickinson) was added to the broth solutions to prepare plates. Media was sterilized by autoclave at 121° C. for 15 minutes. One colony forming unit (CFU) of Salmonella Enteritidis ATCC 13076 was inoculated in duplicate using 25 mL of Luria Bertani (LB) Broth. Cultures were aerobically grown overnight at 37° C. with shaking at 200 rpm (Innova 4230, New Brunswick, Enfield, CT, USA). Overnight cultures of Salmonella Enteritidis were serially diluted in LB broth to produce a sample concentration with approximately 10⁴ CFU/mL. An aliquot of cells (10⁴ CFU/mL) was collected and used to prepare a heat killed control. Cells were heat killed in a thermomixer at 99° C. for 10 minutes and then cooled to room temperature.

100 μL of LB Broth was added to 4 rows (A-D) of columns 2-12 of a 96 well plate. An aliquot of stock CAase was diluted to 0.1 mg/mL using LB for trial 1 and 1.0 mg/mL for trial 2. In trial 1, 200 μL of 0.1 mg/mL CAase was added to column 1 of 4 rows (A-D) the plate. In trial 2, 200 μL of 1.0 mg/mL CAase was added to column 1 of 4 rows (A-D) the plate. The CAase was serially diluted 2-fold across the columns 1-10 using 100 μL transfers (100 μL of solution was removed from column 10 to ensure the volume was consistent with wells 1-9). 100 μL of heat killed cells was added to column 11. 100 μL of live cells (10⁴ CFU/mL) was added to columns 1-10 and 12. The plates were incubated overnight (20 hours) at 37° C. The absorbance at 600 nm was read in a TECAN SAFIRE² microplate reader.

Circular glass coverslips with a diameter or 12 mm were first cleaned with a 3:1 piranha solution for 30 minutes. The slides were sequentially rinsed twice with 50 mL of filter sterilized nanopure water. The glass slides were allowed to dry under aseptic conditions and packed under nitrogen before use.

Biofilms were grown on glass slides using guidance from a method previously described (Wang et al, Front. in Microbiol., (2020), 11:doi:10.3389/fmich.2020.01695). The slides were inoculated under static conditions at 28° C. in 5 mL of an overnight culture of Salmonella Enteritidis. After 3 hours the slides were gently rinsed with LB and resubmerged in 5 mL of LB. The samples were statically incubated at 28° C. The media was carefully decanted and exchanged again after 3 days. After 8 days, the slides were sequentially washed twice in 50 mL of PBS.

### Biofilm Preparation RTE Meat

Raw poultry can be purchased from a local supermarket and aseptically cut into 0.55 g cylindrical samples. The poultry can be sliced to a thickness ranging from 4 to 6 mm and then cut with a #8 corkborer to replicate the diameter of the glass slides, 12 mm. The cylinders can be weighed to select samples that would utilize the ratio of sample to media (25 g/225 mL or 325 g/1625 mL) used in the Microbiology Laboratory Guide method number 8 (United States Department of Agriculture Food Safety and Inspection Service & Science). Biofilms can be grown on the raw poultry samples using the protocol described for sausages. In order to simulate varying degrees of contamination on the meat, 5 mL of the serial dilution and 0.55 g samples can be seeded with a concentration of Salmonella Enteritidis ranging from 10⁷ CFU/mL to 10² CFU/mL. Samples that were not inoculated will serve as negative controls.

Surfaces with Salmonella I were treated with 5 mL PBS, or 5 mL 0.1 mg/mL CAase in PBS. The samples were placed in an incubator (Innova 4230, New Brunswick, Enfield, CT, USA) at 25° C. with shaking at 30 RPM. Liquid above the surfaces (slightly below the meniscus) was collected for bacteria plating, PCR and SEM imaging. After the aliquot was collected during time course trials, the equivalent volume of PBS or 0.1 mg/mL CAase was added to the sample to ensure the volume remained consistent over the course of the experiment.

200 μL sample volumes were collected at times 0, 0.5, 1, 2, 3,4,5 and 6 hours and used to conduct a qPCR assay previously described (Suo et al, supra). Briefly, the 200 μL sample was centrifuged 6600×g for 10 minutes. A pellet was not visible in all the samples so 180 μL of supernatant was discarded and then 20 μL of PrepMan Ultra Reagent (Applied Biosystems, Rotkreuz, Switzerland) was added before thermomixing at 100° C. for 10 minutes. The samples were centrifuged again for 10 minutes at 6600×g and 15 μL of supernatant was removed. Multiplex real-time PCR was performed using a 7500 PCR system (Applied Biosystems). An internal amplification control (IAC) previously reported (Suo et al, supra) was incorporated into the multiplex PCR assay to indicate false-negative results. 2 μL of each sample was added to a well in a 96 well plate containing an 18 μL mixture of 1×TaqMan Gene Expression Master Mix (Applied Biosystems), two sets of primers (invA and IAC) and probes (Cal Fluor Orange 560 and TAMRA) with a concentration of 200 nM each, 1.2×10⁴ copies of IAC and ddH₂O. Real-time PCR was performed in triplicate with the thermocycling: 95° C. for 10 min, followed by 40 cycles of 95° C. for 15 sec, and 60° C. for 1 min. The measured responses were analyzed using automatic Ct and automatic baseline settings in v1.4 of the software and exported to JMP for analysis.

To image biofilms, glass coverslips treated with PBS or CAase for 6 hours were gently rinsed with PBS before being fixed in 2.5% glutaraldehyde. All the quantitative data was analyzed and graphically presented using JMP software version 14.3.0.

### CAase Minimum Inhibitory Concentration (MIC)

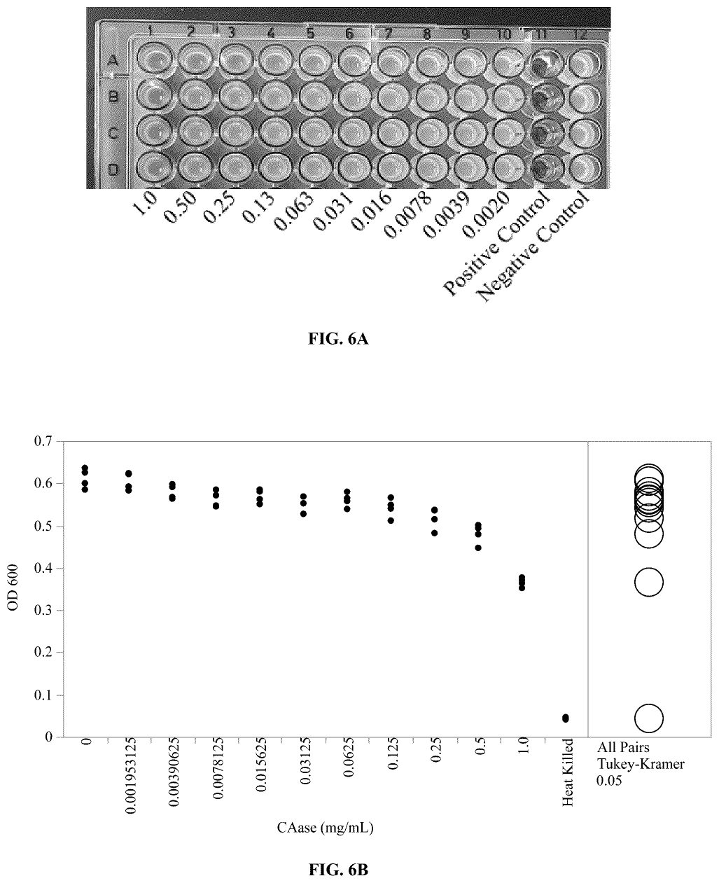

Similar to example 1, the MIC of the enzyme was assessed for Salmonella Enteritidis. Visual inspection of the plate (FIG. 6A) determined the MIC for CAase to be >1.0 mg/mL, which was beyond the range tested in this report. T-tests comparing the optical density measurements at 600 nm (OD₆₀₀) of live cells treated with CAase and heat-killed cells substantiated this conclusion (p<0.0001 for comparing all levels) (FIG. 6B). The OD₆₀₀ values for CAase treatments greater than 0.0078 mg/mL differed significantly from the absence of CAase (p<0.034), suggesting that Salmonella Enteritidis may be more sensitive to CAase than L. monocytogenes. Although growth differences were seen in the presence of CAase compared to its absence, an enzyme concentration of 0.1 mg/mL did not prohibit the growth of Salmonella Enteritidis and was therefore utilized to test the effects of the addition of CAase on detection of Salmonella Enteritidis.

### Detection of Salmonella Enteritidis Biofilms Using qPCR

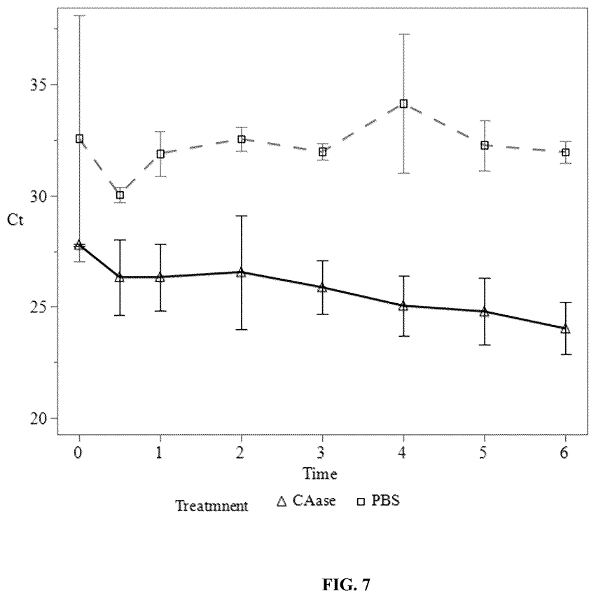

Results from the qPCR-based assay used to detect Salmonella Enteritidis are presented in FIG. 7. Similar to the results presented in Example 1 for L. moncytogenes, it appears that when samples are grown on glass coverslips, CAase treatment (0.1 mg/mL) led to a substantial change in the number of cycles (cycle threshold, Ct) detected (or determined) by qPCR. With the addition of enzyme, Ct values declined from 28 cycles to 24 cycles over the course of 6 hours. In the absence of enzyme there is no statistically different change (p>0.43) in Ct throughout the 6-hour treatment course. This indicates more cells are released into the solution from biofilms treated with CAase than with PBS buffer alone.

### Scanning EM Analysis of Treated Biofilms

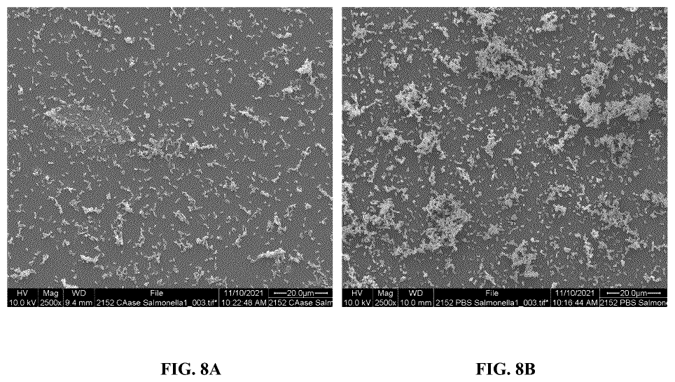

To understand how CAase may be affecting biofilm structure and morphology, biofilms were grown on glass coverslips followed by CAase or PBS treatment for 6 hours, then glutaraldehyde fixed and imaged using scanning electron microscopy (SEM). Similar to L. monocytogenes as presented in Example 1, biofilm morphologies were observed for untreated cells (FIG. 8B), whereas these structures are degraded and removed with sub-MIC CAase treatment (FIG. 8A). At 2,500× magnification, the buffer-only control showed distinct 3-D clusters of cells present on the glass surface throughout each image (FIG. 8B), whereas samples treated with CAase had overall fewer cells present and fewer clusters of cells (FIG. 8A). Consistent with our MIC and biofilm measurements, no significant changes in cell wall integrity were observed. Thus, the SEM results provide further evidence to support a primary role for CAase in removing adherent biofilm of Salmonella Enteritidis.

What is claimed is:

1. A method for identifying microbial cells embedded in a biofilm, comprising the steps of:
a. contacting said biofilm with an enzyme for a sufficient time to release microbial cells from said biofilm;
b. separating released microbial cells from the biofilm material;
c. collecting the released microbial cells; and
d. determining the genus or species of at least one released microbial cell, thereby identifying a microbial cell previously embedded in the biofilm.

2. The method of claim 1, wherein the microbe is a bacterium.

3. The method of claim 2, wherein the bacterium is Listeria monocytogenes or Salmonella Enteritidis.

4. The method of claim 1, wherein the enzyme is CAase.

5. The method of claim 1, wherein the at least one released microbial cell is not contacted with a growth medium.

6. The method of claim 1, wherein all steps are completed within 6 hours.

7. The method of claim 1, wherein the sufficient time is less than 2 hours.

8. A method for identifying microbial cells adhered to a surface, comprising the steps of:
a. contacting said surface with an enzyme for a sufficient time to release microbial cells from said surface;
b. collecting the released microbial cells; and
c. determining the genus or species of at least one released microbial cell, thereby identifying a microbial cell previously adhered to said surface.

9. The method of claim 8, wherein the microbe is a bacterium.

10. The method of claim 8, wherein the enzyme is CAase.

11. The method of claim 8, wherein the at least one released microbial cell is not contacted with a growth medium.

12. The method of claim 8, wherein the surface is a biotic surface.

13. The method of claim 12, wherein the biotic surface is meat, a ready-to-eat meat product, or fresh produce.

14. The method of claim 8, wherein the surface is an abiotic surface.

15. The method of claim 14, wherein the abiotic surface is a food preparation surface.

16. The method of claim 8, wherein all steps are completed within 6 hours.

17. The method of claim 8, wherein the sufficient time is less than 2 hours.
